(19) United States
(12) Patent Application Publication
(10) Pub. No.: US 2019/0159704 A1
(43) Pub. Date: May 30, 2019

(54) EXTENDING BATTERY LIFE

(71) Applicant: Senseonics, Incorporated, Germantown, MD (US)

(72) Inventors: Rober Matikyan, Germantown, MD (US); Todd Coram, Germantown, MD (US)

(73) Assignee: Senseonics, Incorporated, Germantown, MD (US)

(21) Appl. No.: 16/199,918

(22) Filed: Nov. 26, 2018

Related U.S. Application Data
(60) Provisional application No. 62/590,822, filed on Nov. 27, 2017.

(51) Int. Cl. A61B 5/145; G01R 31/392; H01M 10/48

(52) U.S. Cl. CPC A61B 5/14532; H01M 10/48; G01R 31/392

(57) ABSTRACT

A system and method of extending the life of a battery. The system may include a display device and a transceiver. The transceiver may include the battery. The transceiver may be configured to perform a battery level reading function to determine a battery level of the battery. The transceiver may be configured to time performance of the battery level reading function such that it does not occur at the same time as a period of high current demand on the battery. The transceiver may be capable of executing concurrently two or more of the functions, and the transceiver may be configured to avoid executing concurrently one or more of: (a) a number of functions greater than a maximum number of functions; (b) functions including one or more high current demand functions; and (c) two or more functions that would have a combined current demand higher than a current demand threshold.

### CROSS-REFERENCE TO RELATED APPLICATION

The present application claims the benefit of priority to U.S. Provisional Application Ser. No. 62/590,822, filed on Nov. 27, 2017, which is incorporated herein by reference in its entirety.

### BACKGROUND

### Field of Invention

Aspects of the present invention may relate to methods and systems for extending battery life. More specifically, some aspects of the present invention may relate to extending the life of a battery in a transceiver of an analyte monitoring system.

### Discussion of the Background

The prevalence of diabetes mellitus continues to increase in industrialized countries, and projections suggest that this figure will rise to 4.4% of the global population (366 million individuals) by the year 2030. Glycemic control is a key determinant of long-term outcomes in patients with diabetes, and poor glycemic control is associated with retinopathy, nephropathy and an increased risk of myocardial infarction, cerebrovascular accident, and peripheral vascular disease requiring limb amputation. Despite the development of new insulins and other classes of antidiabetic therapy, roughly half of all patients with diabetes do not achieve recommended target hemoglobin A1c (HbA1c) levels <7.0%.

Frequent self-monitoring of blood glucose (SMBG) is necessary to achieve tight glycemic control in patients with diabetes mellitus, particularly for those requiring insulin therapy. However, current blood (finger-stick) glucose tests are burdensome, and, even in structured clinical studies, patient adherence to the recommended frequency of SMBG decreases substantially over time. Moreover, finger-stick measurements only provide information about a single point in time and do not yield information regarding intraday fluctuations in blood glucose levels that may more closely correlate with some clinical outcomes.

Continuous glucose monitors (CGMs) have been developed in an effort to overcome the limitations of finger-stick SMBG and thereby help improve patient outcomes. These systems enable increased frequency of glucose measurements and a better characterization of dynamic glucose fluctuations, including episodes of unrealized hypoglycemia. Furthermore, integration of CGMs with automated insulin pumps allows for establishment of a closed-loop “artificial pancreas” system to more closely approximate physiologic insulin delivery and to improve adherence.

Monitoring real-time analyte measurements from a living body via wireless analyte monitoring sensor(s) may provide numerous health and research benefits. There is a need to enhance such analyte monitoring systems via innovations.

### SUMMARY

One aspect of the invention may provide an analyte monitoring system including a display device and a transceiver. The transceiver may include a battery and may be configured to (i) perform a battery level reading function to determine a battery level of the battery and (ii) convey to the display device one or more of the determined battery level and a battery level alert, alarm, or notification generated based the determined battery level. The processor may be configured to time performance of the battery level reading function such that it does not occur at the same time as a period of high current demand on the battery.

In some embodiments, timing performance of the battery level reading function such that it does not occur at the same time as the period of high current demand on the battery may include not performing the battery level reading function at the same time as the transceiver is executing concurrently a threshold number of functions or more. In some embodiments, timing performance of the battery level reading function such that it does not occur at the same time as the period of high current demand on the battery may include not performing the battery level reading function at the same time as the transceiver is executing one or more high current demand functions. In some embodiments, the system may further include an analyte sensor, and the one or more high current demand functions may include communicating with the analyte sensor.

In some embodiments, timing performance of the battery level reading function such that it does not occur at the same time as the period of high current demand on the battery may include not performing the battery level reading function at the same time as the transceiver is executing concurrently functions that have a combined current demand higher than a current demand threshold. In some embodiments, timing performance of the battery level reading function such that it does not occur at the same time as the period of high current demand on the battery may include using one or more software semaphores.

In some embodiments, performing the battery level reading function may include sampling the voltage of the battery. In some embodiments, the processor may be further configured to time performance of the battery level reading function such that it does not occur soon after the period of high current demand on the battery. In some embodiments, the processor may be further configured to perform the battery level reading function in a time frame isolated from the period of high current demand such that enough time passes after the period of high current demand for the battery level reading to be accurate and not reflect a temporary voltage drop due to the high current demand.

Another aspect of the invention may provide an analyte monitoring system including an analyte sensor, a display device, and a transceiver. The transceiver may include a battery. The transceiver may be configured to (1) execute functions including communicating with the analyte sensor and communicating with the display device and (2) be capable of executing concurrently two or more of the functions. The transceiver may be configured to (3) avoid executing concurrently one or more of: (a) a number of functions greater than a maximum number of functions, (b) functions including one or more high current demand functions, and (c) two or more functions that would have a combined current demand higher than a current demand threshold.

In some embodiments, the transceiver may be configured to avoid executing concurrently the function of communicating with the analyte sensor and another of the functions. In some embodiments, the transceiver may be configured to avoid executing concurrently the functions of communicating with the analyte sensor and communicating with the display device. In some embodiments, the transceiver may be configured to (i) perform a battery level reading function to determine a battery level of the battery and (ii) convey to the display device one or more of the determined battery level and a battery level alert, alarm, or notification generated based the determined battery level. The processor may be configured to time performance of the battery level reading function such that it does not occur at the same time as a period of high current demand on the battery.

Still another aspect of the invention may provide a method. The method may include using a transceiver to perform a battery level reading function to determine a battery level of a battery of the transceiver. The method may include using the transceiver to convey to a display device one or more of the determined battery level and a battery level alert, alarm, or notification generated based the determined battery level. The method may include using the transceiver to time performance of the battery level reading function such that it does not occur at the same time as a period of high current demand on the battery.

Yet another aspect of the invention may provide a method. The method may include using transceiver to execute functions. The method may include using the transceiver to concurrently execute two or more of the functions. The method may include using the transceiver to avoid executing concurrently one or more of: (a) a number of functions greater than a maximum number of functions, (b) functions including one or more high current demand functions, and (c) two or more functions that would have a combined current demand higher than a current demand threshold. In some embodiments, the functions may include communicating with an analyte sensor and communicating with a display device.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 is a schematic view illustrating an analyte monitoring system embodying aspects of the present invention.

FIG. 2 is a schematic view illustrating a sensor and transceiver of an analyte monitoring system embodying aspects of the present invention.

FIG. 3 is cross-sectional, perspective view of a transceiver embodying aspects of the invention.

FIG. 4 is an exploded, perspective view of a transceiver embodying aspects of the invention.

FIG. 5 is a schematic view illustrating a transceiver embodying aspects of the present invention.

### DETAILED DESCRIPTION OF PREFERRED EMBODIMENTS

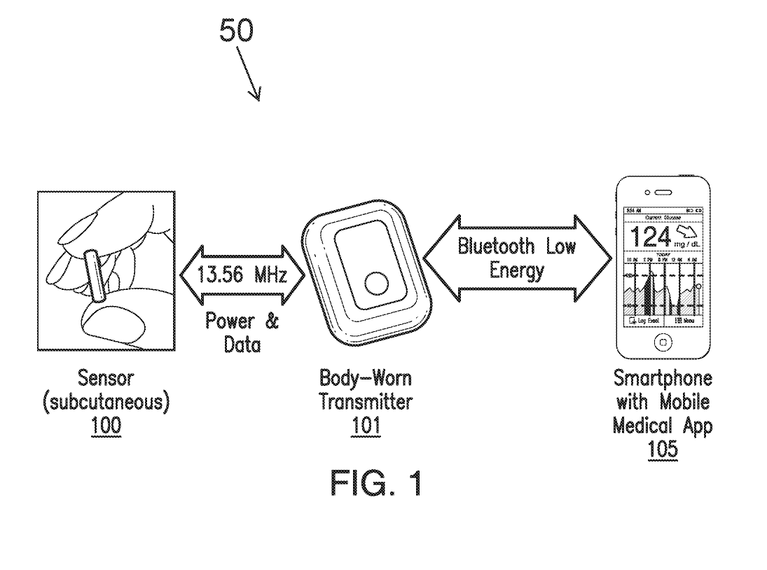

FIG. 1 is a schematic view of an exemplary analyte monitoring system 50 embodying aspects of the present invention. The analyte monitoring system 50 may be a continuous analyte monitoring system (e.g., a continuous glucose monitoring system). In some embodiments, the analyte monitoring system 50 may include one or more of an analyte sensor 100, a transceiver 101, and a display device 105. In some embodiments, the sensor 100 may be small, fully subcutaneously implantable sensor measures analyte (e.g., glucose) concentrations in a medium (e.g., interstitial fluid) of a living animal (e.g., a living human). However, this is not required, and, in some alternative embodiments, the sensor 100 may be a partially implantable (e.g., transcutaneous) sensor or a fully external sensor. In some embodiments, the transceiver 101 may be an externally worn transceiver (e.g., attached via an armband, wristband, waistband, or adhesive patch). In some embodiments, the transceiver 101 may remotely power and/or communicate with the sensor to initiate and receive the measurements (e.g., via near field communication (NFC)). However, this is not required, and, in some alternative embodiments, the transceiver 101 may power and/or communicate with the sensor 100 via one or more wired connections. In some non-limiting embodiments, the transceiver 101 may be a smartphone (e.g., an NFC-enabled smartphone). In some embodiments, the transceiver 101 may communicate information (e.g., one or more analyte concentrations) wirelessly (e.g., via a Bluetooth™ communication standard such as, for example and without limitation Bluetooth Low Energy) to a hand held application running on a display device 105 (e.g., smartphone). In some embodiments, information can be downloaded from the transceiver 101 through a Universal Serial Bus (USB) port. In some embodiments, the analyte monitoring system 50 may include a web interface for plotting and sharing of uploaded data.

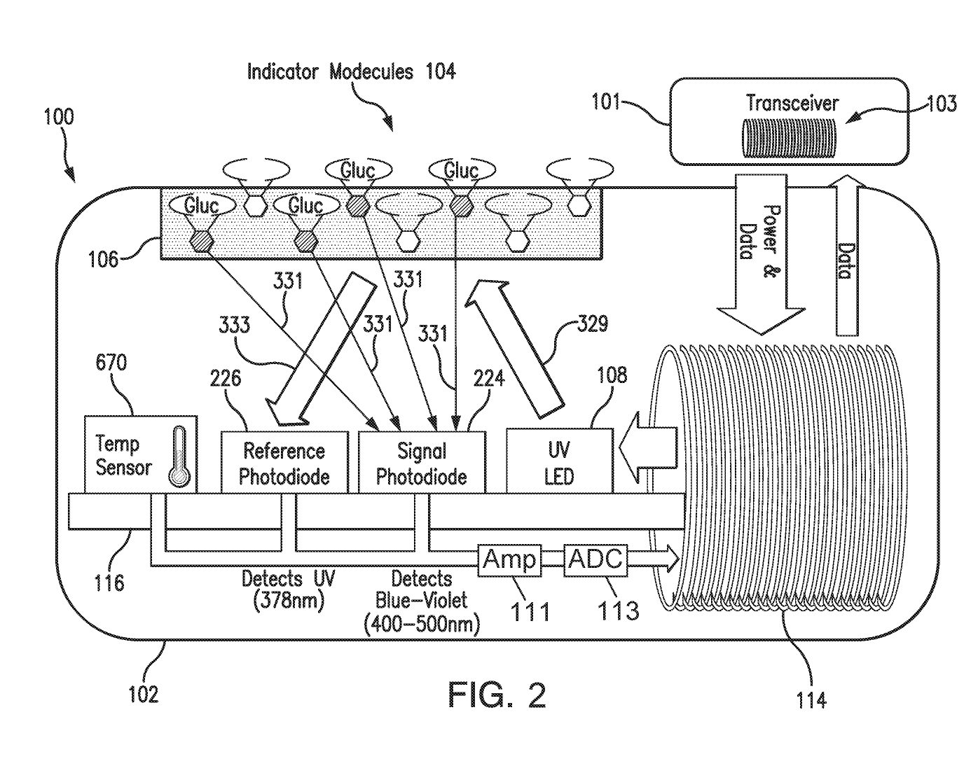

In some embodiments, as illustrated in FIG. 2, the transceiver 101 may include an inductive element 103, such as, for example, a coil. The transceiver 101 may generate an electromagnetic wave or electrodynamic field (e.g., by using a coil) to induce a current in an inductive element 114 of the sensor 100, which powers the sensor 100. The transceiver 101 may also convey data (e.g., commands) to the sensor 100. For example, in a non-limiting embodiment, the transceiver 101 may convey data by modulating the electromagnetic wave used to power the sensor 100 (e.g., by modulating the current flowing through a coil 103 of the transceiver 101). The modulation in the electromagnetic wave generated by the transceiver 101 may be detected/extracted by the sensor 100. Moreover, the transceiver 101 may receive data (e.g., measurement information) from the sensor 100. For example, in a non-limiting embodiment, the transceiver 101 may receive data by detecting modulations in the electromagnetic wave generated by the sensor 100, e.g., by detecting modulations in the current flowing through the coil 103 of the transceiver 101.

The inductive element 103 of the transceiver 101 and the inductive element 114 of the sensor 100 may be in any configuration that permits adequate field strength to be achieved when the two inductive elements are brought within adequate physical proximity.

In some non-limiting embodiments, as illustrated in FIG. 2, the sensor 100 may be encased in a sensor housing 102 (i.e., body, shell, capsule, or encasement), which may be rigid and biocompatible. The sensor 100 may include an analyte indicator element 106, such as, for example, a polymer graft coated, diffused, adhered, or embedded on or in at least a portion of the exterior surface of the sensor housing 102. The analyte indicator element 106 (e.g., polymer graft) of the sensor 100 may include indicator molecules 104 (e.g., fluorescent indicator molecules) exhibiting one or more detectable properties (e.g., optical properties) based on the amount or concentration of the analyte in proximity to the analyte indicator element 106. In some embodiments, the sensor 100 may include a light source 108 that emits excitation light 329 over a range of wavelengths that interact with the indicator molecules 104. The sensor 100 may also include one or more photodetectors 224, 226 (e.g., photodiodes, phototransistors, photoresistors, or other photosensitive elements). The one or more photodetectors (e.g., photodetector 224) may be sensitive to emission light 331 (e.g., fluorescent light) emitted by the indicator molecules 104 such that a signal generated by a photodetector (e.g., photodetector 224) in response thereto that is indicative of the level of emission light 331 of the indicator molecules and, thus, the amount of analyte of interest (e.g., glucose). In some non-limiting embodiments, one or more of the photodetectors (e.g., photodetector 226) may be sensitive to excitation light 329 that is reflected from the analyte indicator element 106 as reflection light 333. In some non-limiting embodiments, one or more of the photodetectors may be covered by one or more filters (e.g., bandpass filter 112 of FIG. 6) that allow only a certain subset of wavelengths of light to pass through (e.g., a subset of wavelengths corresponding to emission light 331 or a subset of wavelengths corresponding to reflection light 333) and reflect the remaining wavelengths. In some non-limiting embodiments, the sensor 100 may include a temperature transducer 670. In some non-limiting embodiments, the sensor 100 may include a drug-eluting polymer matrix that disperses one or more therapeutic agents (e.g., an anti-inflammatory drug).

In some embodiments, the outputs of one or more of the photodetectors 224, 226 and the temperature transducer 670 may be amplified by an amplifier 111. In some non-limiting embodiments, the amplifier 111 may be a comparator that receives analog light measurement signals from the photodetectors 224, 226 and output an analog light difference measurement signal indicative of the difference between the received analog light measurement signals. In some non-limiting embodiments, the amplifier 111 may be a transimpedance amplifier. However, in some alternative embodiments, a different amplifier may be used. In some embodiments, the outputs of one or more of the photodetectors 224, 226, the temperature transducer 670, and the amplifier 111 may be converted to a digital signal by an analog-to-digital converter (ADC) 113.

In some embodiments, one or more of the gain of the amplifier 111 and the drive current of the light source 108 may be initially set during a quality control process. In some embodiments, one or more of the gain of the amplifier 111 and the drive current of the light source 108 may be set to allow high dynamic range and to keep the modulated signal within the operational region. In some embodiments, any change (e.g., increase or decrease) to one or more of the drive current of the light source 108 and the gain of the amplifier 111 may change the modulated signal level accordingly.

In some embodiments, as illustrated in FIG. 2, the sensor 100 may include a substrate 116. In some embodiments, the substrate 116 may be a circuit board (e.g., a printed circuit board (PCB) or flexible PCB) on which circuit components (e.g., analog and/or digital circuit components) may be mounted or otherwise attached. However, in some alternative embodiments, the substrate 116 may be a semiconductor substrate having circuitry fabricated therein. The circuitry may include analog and/or digital circuitry. Also, in some semiconductor substrate embodiments, in addition to the circuitry fabricated in the semiconductor substrate, circuitry may be mounted or otherwise attached to the semiconductor substrate 116. In other words, in some semiconductor substrate embodiments, a portion or all of the circuitry, which may include discrete circuit elements, an integrated circuit (e.g., an application specific integrated circuit (ASIC)) and/or other electronic components (e.g., a non-volatile memory), may be fabricated in the semiconductor substrate 116 with the remainder of the circuitry is secured to the semiconductor substrate 116 and/or a core (e.g., ferrite core) for the inductive element 114. In some embodiments, the semiconductor substrate 116 and/or a core may provide communication paths between the various secured components.

In some embodiments, the one or more of the sensor housing 102, analyte indicator element 106, indicator molecules 104, light source 108, photodetectors 224, 226, temperature transducer 670, substrate 116, and inductive element 114 of sensor 100 may include some or all of the features described in one or more of U.S. application Ser. No. 13/761,839, filed on Feb. 7, 2013, U.S. application Ser. No. 13/937,871, filed on Jul. 9, 2013, and U.S. application Ser. No. 13/650,016, filed on Oct. 11, 2012, all of which are incorporated by reference in their entireties. Similarly, the structure and/or function of the sensor 100 and/or transceiver 101 may be as described in one or more of U.S. application Ser. Nos. 13/761,839, 13/937,871, and 13/650,016.

Although in some embodiments, as illustrated in FIG. 2, the sensor 100 may be an optical sensor, this is not required, and, in one or more alternative embodiments, sensor 100 may be a different type of analyte sensor, such as, for example, an electrochemical sensor, a diffusion sensor, or a pressure sensor. Also, although in some embodiments, as illustrated in FIGS. 1 and 2, the analyte sensor 100 may be a fully implantable sensor, this is not required, and, in some alternative embodiments, the sensor 100 may be a transcutaneous sensor having a wired connection to the transceiver 101. For example, in some alternative embodiments, the sensor 100 may be located in or on a transcutaneous needle (e.g., at the tip thereof). In these embodiments, instead of wirelessly communicating using inductive elements 103 and 114, the sensor 100 and transceiver 101 may communicate using one or more wires connected between the transceiver 101 and the transceiver transcutaneous needle that includes the sensor 100. For another example, in some alternative embodiments, the sensor 100 may be located in a catheter (e.g., for intravenous blood glucose monitoring) and may communicate (wirelessly or using wires) with the transceiver 101.

In some embodiments, the sensor 100 may include a transceiver interface device. In some embodiments where the sensor 100 includes an antenna (e.g., inductive element 114), the transceiver interface device may include the antenna (e.g., inductive element 114) of sensor 100. In some of the transcutaneous embodiments where there exists a wired connection between the sensor 100 and the transceiver 101, the transceiver interface device may include the wired connection.

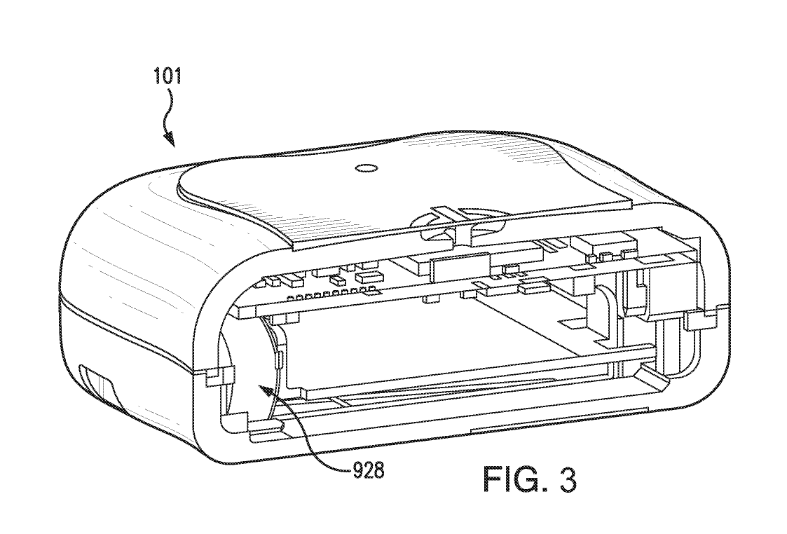

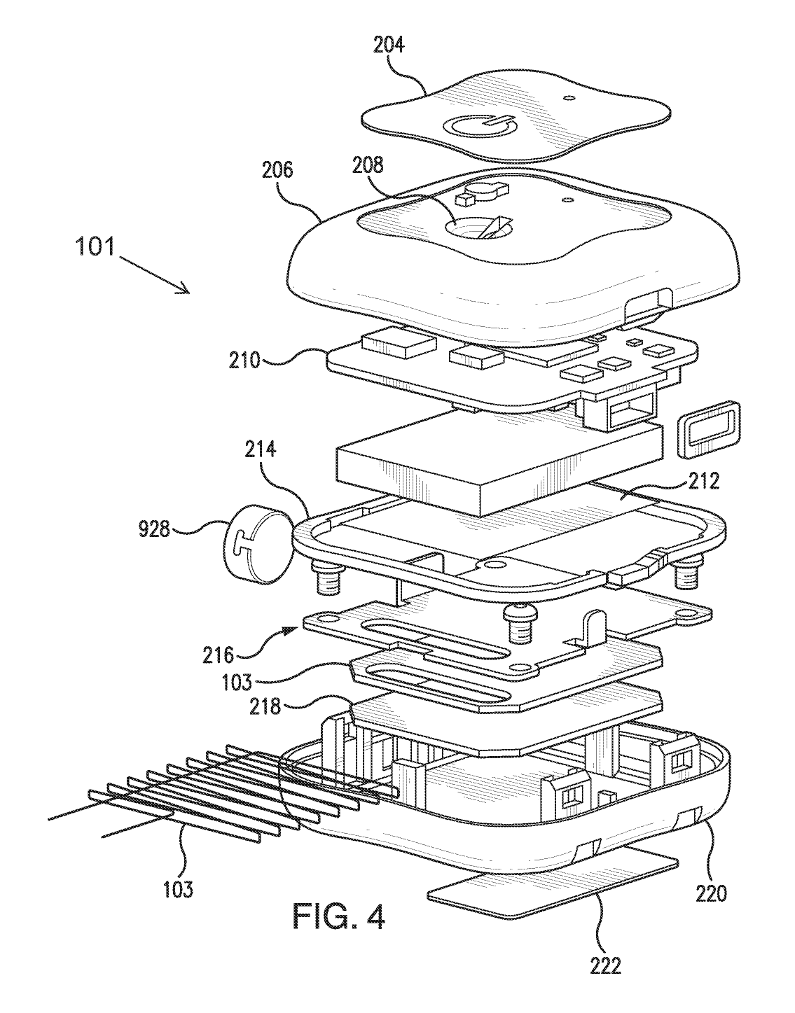

FIGS. 3 and 4 are cross-sectional and exploded views, respectively, of a non-limiting embodiment of the transceiver 101, which may be included in the analyte monitoring system illustrated in FIG. 1. As illustrated in FIG. 4, in some non-limiting embodiments, the transceiver 101 may include a graphic overlay 204, front housing 206, button 208, printed circuit board (PCB) assembly 210, battery 212, gaskets 214, antenna 103, frame 218, reflection plate 216, back housing 220, ID label 222, and/or vibration motor 928. In some non-limiting embodiments, the vibration motor 928 may be attached to the front housing 206 or back housing 220 such that the battery 212 does not dampen the vibration of vibration motor 928. In a non-limiting embodiment, the transceiver electronics may be assembled using standard surface mount device (SMD) reflow and solder techniques. In one embodiment, the electronics and peripherals may be put into a snap together housing design in which the front housing 206 and back housing 220 may be snapped together. In some embodiments, the full assembly process may be performed at a single external electronics house. However, this is not required, and, in alternative embodiments, the transceiver assembly process may be performed at one or more electronics houses, which may be internal, external, or a combination thereof. In some embodiments, the assembled transceiver 101 may be programmed and functionally tested. In some embodiments, assembled transceivers 101 may be packaged into their final shipping containers and be ready for sale.

In some embodiments, as illustrated in FIGS. 3 and 4, the antenna 103 may be contained within the housing 206 and 220 of the transceiver 101. In some embodiments, the antenna 103 in the transceiver 101 may be small and/or flat so that the antenna 103 fits within the housing 206 and 220 of a small, lightweight transceiver 101. In some embodiments, the antenna 103 may be robust and capable of resisting various impacts. In some embodiments, the transceiver 101 may be suitable for placement, for example, on an abdomen area, upper-arm, wrist, or thigh of a patient body. In some non-limiting embodiments, the transceiver 101 may be suitable for attachment to a patient body by means of a biocompatible patch. Although, in some embodiments, the antenna 103 may be contained within the housing 206 and 220 of the transceiver 101, this is not required, and, in some alternative embodiments, a portion or all of the antenna 103 may be located external to the transceiver housing. For example, in some alternative embodiments, antenna 103 may wrap around a user's wrist, arm, leg, or waist such as, for example, the antenna described in U.S. Pat. No. 8,073,548, which is incorporated herein by reference in its entirety.

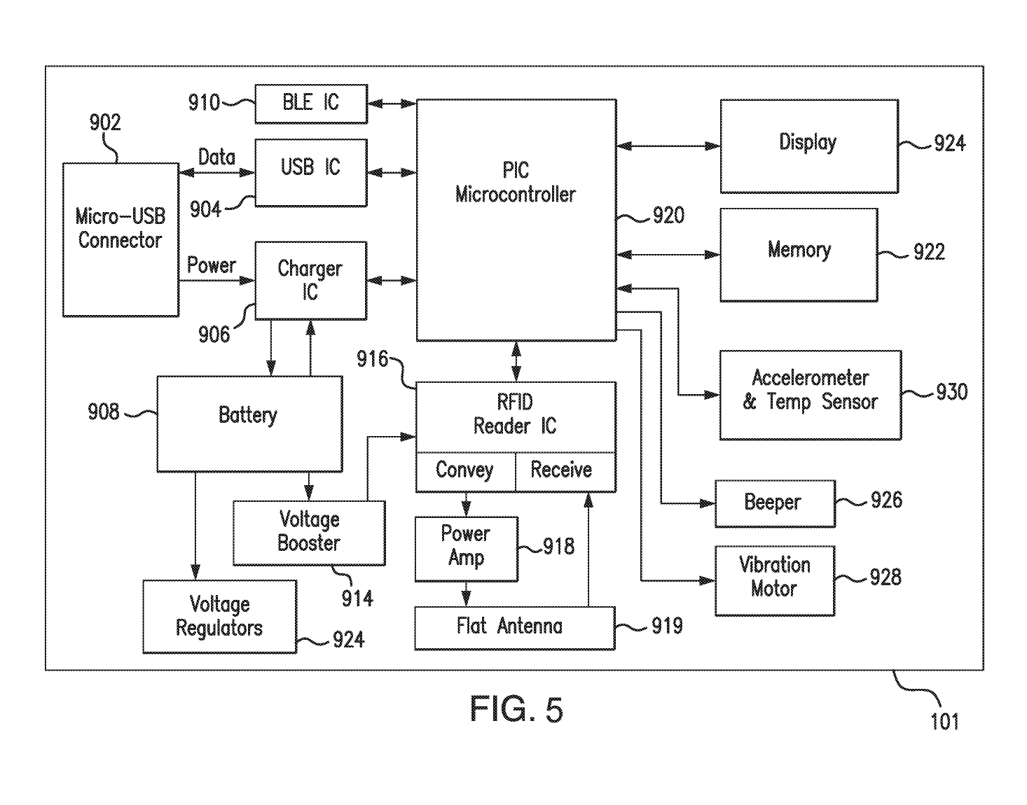

FIG. 5 is a schematic view of an external transceiver 101 according to a non-limiting embodiment. In some embodiments, the transceiver 101 may have a connector 902, such as, for example, a Micro-Universal Serial Bus (USB) connector. The connector 902 may enable a wired connection to an external device, such as a personal computer (e.g., personal computer 109) or a display device 105 (e.g., a smartphone).

The transceiver 101 may exchange data to and from the external device through the connector 902 and/or may receive power through the connector 902. The transceiver 101 may include a connector integrated circuit (IC) 904, such as, for example, a USB-IC, which may control transmission and receipt of data through the connector 902. The transceiver 101 may also include a charger IC 906, which may receive power via the connector 902 and charge a battery 908 (e.g., lithium-polymer battery). In some embodiments, the battery 908 may be rechargeable, may have a short recharge duration, and/or may have a small size.

In some embodiments, the transceiver 101 may include one or more connectors in addition to (or as an alternative to) Micro-USB connector 904. For example, in one alternative embodiment, the transceiver 101 may include a spring-based connector (e.g., Pogo pin connector) in addition to (or as an alternative to) Micro-USB connector 904, and the transceiver 101 may use a connection established via the spring-based connector for wired communication to a personal computer (e.g., personal computer 109) or a display device 105 (e.g., a smartphone) and/or to receive power, which may be used, for example, to charge the battery 908.

In some embodiments, the transceiver 101 may have a wireless communication IC 910, which enables wireless communication with an external device, such as, for example, one or more personal computers (e.g., personal computer 109) or one or more display devices 105 (e.g., a smartphone). In one non-limiting embodiment, the wireless communication IC 910 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some non-limiting embodiments, the wireless communication IC 910 may be configured to wirelessly transmit data at a frequency greater than 1 gigahertz (e.g., 2.4 or 5 GHz). In some embodiments, the wireless communication IC 910 may include an antenna (e.g., a Bluetooth antenna). In some non-limiting embodiments, the antenna of the wireless communication IC 910 may be entirely contained within the housing (e.g., housing 206 and 220) of the transceiver 101. However, this is not required, and, in alternative embodiments, all or a portion of the antenna of the wireless communication IC 910 may be external to the transceiver housing.

In some embodiments, the transceiver 101 may include a display interface device, which may enable communication by the transceiver 101 with one or more display devices 105. In some embodiments, the display interface device may include the antenna of the wireless communication IC 910 and/or the connector 902. In some non-limiting embodiments, the display interface device may additionally include the wireless communication IC 910 and/or the connector IC 904.

In some embodiments, the transceiver 101 may include voltage regulators 912 and/or a voltage booster 914. The battery 908 may supply power (via voltage booster 914) to radio-frequency identification (RFID) reader IC 916, which uses the inductive element 103 to convey information (e.g., commands) to the sensor 101 and receive information (e.g., measurement information) from the sensor 100. In some non-limiting embodiments, the sensor 100 and transceiver 101 may communicate using near field communication (NFC) (e.g., at a frequency of 13.56 MHz). In the illustrated embodiment, the inductive element 103 is a flat antenna. In some non-limiting embodiments, the antenna may be flexible. However, as noted above, the inductive element 103 of the transceiver 101 may be in any configuration that permits adequate field strength to be achieved when brought within adequate physical proximity to the inductive element 114 of the sensor 100. In some embodiments, the transceiver 101 may include a power amplifier 918 to amplify the signal to be conveyed by the inductive element 103 to the sensor 100.

The transceiver 101 may include a processor 920 and a memory 922 (e.g., Flash memory). In some non-limiting embodiments, the memory 922 may be non-volatile and/or capable of being electronically erased and/or rewritten. In some non-limiting embodiments, the processor 920 may be, for example and without limitation, a peripheral interface controller (PIC) microcontroller. In some embodiments, the processor 920 may control the overall operation of the transceiver 101. For example, the processor 920 may control the connector IC 904 or wireless communication IC 910 to transmit data via wired or wireless communication and/or control the RFID reader IC 916 to convey data via the inductive element 103. The processor 920 may also control processing of data received via the inductive element 103, connector 902, or wireless communication IC 910.

In some embodiments, the transceiver 101 may include a sensor interface device, which may enable communication by the transceiver 101 with a sensor 100. In some embodiments, the sensor interface device may include the inductive element 103. In some non-limiting embodiments, the sensor interface device may additionally include the RFID reader IC 916 and/or the power amplifier 918. However, in some alternative embodiments where there exists a wired connection between the sensor 100 and the transceiver 101 (e.g., transcutaneous embodiments), the sensor interface device may include the wired connection.

In some embodiments, the transceiver 101 may include a display 924 (e.g., liquid crystal display and/or one or more light emitting diodes), which the processor 920 may control to display data (e.g., analyte concentration values). In some embodiments, the transceiver 101 may include a speaker 926 (e.g., a beeper) and/or vibration motor 928, which may be activated, for example, in the event that an alarm condition (e.g., detection of a hypoglycemic or hyperglycemic condition) is met. The transceiver 101 may also include one or more additional sensors 930, which may include an accelerometer and/or temperature sensor that may be used in the processing performed by the processor 920.

In some embodiments, the transceiver 101 may be a body-worn transceiver that is a rechargeable, external device worn over the sensor implantation or insertion site. The transceiver 101 may supply power to the proximate sensor 100, calculate analyte concentrations from data received from the sensor 100, and/or transmit the calculated analyte concentrations to a display device 105 (see FIG. 1). Power may be supplied to the sensor 100 through an inductive link (e.g., an inductive link of 13.56 MHz). In some embodiments, the transceiver 101 may be placed using an adhesive patch or a specially designed strap or belt. The external transceiver 101 may read measured analyte data from a subcutaneous sensor 100 (e.g., up to a depth of 2 cm or more). The transceiver 101 may periodically (e.g., every 2, 5, or 10 minutes) read sensor data and calculate an analyte concentration and an analyte concentration trend. From this information, the transceiver 101 may also determine if an alert and/or alarm condition exists, which may be signaled to the user (e.g., through vibration by vibration motor 928 and/or an LED of the transceiver's display 924 and/or a display of a display device 105). The information from the transceiver 101 (e.g., calculated analyte concentrations, calculated analyte concentration trends, alerts, alarms, and/or notifications) may be transmitted to a display device 105 (e.g., via Bluetooth Low Energy with Advanced Encryption Standard (AES)-Counter CBC-MAC (CCM) encryption) for display by a mobile medical application (MMA) being executed by the display device 105. In some non-limiting embodiments, the MMA may provide alarms, alerts, and/or notifications in addition to any alerts, alarms, and/or notifications received from the transceiver 101. In one embodiment, the MMA may be configured to provide push notifications. In some embodiments, the transceiver 101 may have a power button (e.g., button 208) to allow the user to turn the device on or off, reset the device, or check the remaining battery life. In some embodiments, the transceiver 101 may have a button, which may be the same button as a power button or an additional button, to suppress one or more user notification signals (e.g., vibration, visual, and/or audible) of the transceiver 101 generated by the transceiver 101 in response to detection of an alert or alarm condition.

In some embodiments, the transceiver 101 of the analyte monitoring system 50 receives raw signals indicative of an amount or concentration of an analyte in proximity to the analyte indicator element 106 of the analyte sensor 100. In some embodiments, the transceiver 101 may receive the raw signals from the sensor 100 periodically (e.g., every 5, 10, or 20 minutes). In some embodiments, the raw signals may include one or more analyte measurements (e.g., one or more measurements indicative of the level of emission light 331 from the indicator molecules 104 as measured by the photodetector 224) and/or one or more temperature measurements (e.g., as measured by the temperature transducer 670). In some embodiments, the transceiver 101 may use the received raw signals to calculate analyte concentration. In some embodiments, the transceiver 100 may store one or more calculated analyte concentrations (e.g., in memory 922). In some embodiments, the transceiver 100 may convey one or more calculated analyte concentrations to the display device 105.

In some embodiments, the analyte monitoring system 50 may calibrate the conversion of raw signals to analyte concentration. In some embodiments, the calibration may be performed approximately periodically (e.g., every 12 or 24 hours). In some embodiments, the calibration may be performed using one or more reference measurements (e.g., one or more self-monitoring blood glucose (SMBG) measurements), which may be entered into the analyte monitoring system 50 using the user interface of the display device 105. In some embodiments, the transceiver 101 may receive the one or more reference measurements from the display device 105 and perform the calibration.

In some embodiments, the transceiver 101 (e.g., the processor 920 of the transceiver 101) may by capable of executing concurrently two or more functions (e.g., threads or processes). For example, in some non-limiting embodiments, the transceiver 101 may execute concurrently two or more of the following functions: (i) communicating with the analyte sensor 100 (e.g., using the voltage booster 914, RFID reader IC 916, power amplifier 918, and inductive element 103), (ii) vibrating the transceiver 101 (e.g., using the vibration motor 928), (iii) turning on the display 924, and (iv) communicating with one or more remote devices (e.g., the display device 105 and/or a personal computer) using one or more of the wireless communication IC 910 and the connector IC 904.

In some embodiments, concurrent execution of multiple functions may place a high current demand on the battery 908 of the transceiver 101 due to the cumulative current consumption of functions being executed at the same time. In some non-limiting embodiments, a high current demand on the battery 908 due to the transceiver 101 executing two or more functions concurrently may have a negative impact for overall battery life compared to the impact of the functions being executed at different times. In some non-limiting embodiments, the negative impact may be that the concurrent execution of multiple functions will drain the battery 908 at a faster rate than if the functions were executed sequentially.

In some embodiments, the transceiver 101 (e.g., the processor 920 of the transceiver 101) may be configured to avoid concurrent execution of multiple transceiver functions. In some embodiments, the transceiver 101 may time the execution of transceiver functions to avoid concurrent execution. In some non-limiting embodiments, the transceiver 101 may be configured to execute concurrently no more than a maximum number of functions. In some non-limiting embodiments, the maximum number of concurrently executed functions may be, for example and without limitation, ten, six, five, four, three, or two. In some alternative embodiments, the transceiver 101 may be configured to avoid any concurrent execution of functions. In some other alternative embodiments, the transceiver 101 may be configured to avoid concurrent execution of certain functions (e.g., high current demand functions). For example and without limitation, the transceiver 101 may be configured to avoid concurrent execution of sensor communication with any other transceiver function. For another example, the transceiver 101 may be configured to avoid concurrent execution of sensor communication and wireless communication with a remote device (e.g., the display device 105). In some additional alternative embodiments, the transceiver 101 may be configured to avoid executing concurrently functions that would have a combined current demand higher than a current demand threshold. For example and without limitation, the transceiver 101 may allow concurrent execution of five functions whose combined current demand is lower than the current demand threshold but would avoid concurrent execution of two functions having a combined current demand higher than the current demand threshold.

In some embodiments, the transceiver 101 may execute a battery level reading function (e.g., a battery level reading thread or a battery level reading process). In some non-limiting embodiments, execution of the battery level reading function may include sampling the voltage of the battery 908. In some non-limiting embodiments, the transceiver 101 may perform the battery level reading function periodically.

In some embodiments, if the transceiver 101 executes the battery level reading function at the same time as (or soon after) high current demand on the battery 908 (e.g., due to concurrent execution of two or more functions or execution of one or more high current demand functions), the battery level reading function may produce a battery level reading that is lower than what the battery level reading would be after the battery 908 has a chance to recover from the high current demand. In some embodiments, the transceiver 101 may convey the lower battery level reading to the display device 105 for display to a user. In some embodiments, based on the lower battery level reading, the transceiver 101 may generate (and convey to the display device 105) a low battery level alert, alarm, or notification indicating that the battery 908 of the transceiver 101 needs charging. Conveyance of the lower battery level reading and/or low battery level alert, alarm, or notification to the display device 105 may result in the user taking action to recharge the battery 908 of the transceiver 101 earlier than needed.

In some embodiments, the transceiver 101 (e.g., the processor 920 of the transceiver 101) may avoid executing the battery level reading function at the same time as (and/or soon after) a period of high current demand on the battery 908. In some non-limiting embodiments, the transceiver 101 may avoid executing the battery level reading function at the same time as (and/or soon after) concurrent execution of a threshold number of functions or more. In some embodiments, the threshold number of functions may be, for example and without limitation, ten, ten, six, five, four, three, or two. In some non-limiting embodiments, the transceiver 101 may additionally or alternatively avoid executing the battery level reading function at the same time as (and/or soon after) execution of one or more high current demand functions. In some embodiments, a high current demand function may be, for example and without limitation, communicating with the analyte sensor 100. In some non-limiting embodiments, the transceiver 101 may additionally or alternatively avoid executing the battery level reading function at the same time as (and/or soon after) concurrent execution of functions that have a combined current demand higher than a current demand threshold.

In some non-limiting embodiments, the transceiver 101 may avoid executing the battery level reading function at the same time as (and/or soon after) a period of high current demand on the battery 908 using one or more software semaphores. In some non-limiting embodiments, the transceiver 101 may execute the battery level reading function in a time frame isolated from one or more high current demand time frames such that enough time passes after a period of high current demand on the battery 908 for the battery level reading to be accurate and not reflect a temporary voltage drop due to the high current demand.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention. For instance, although this invention has been described in the context of a extending the life (and obtaining accurate readings) of a battery in a transceiver of an analyte monitoring system, the invention is applicable to extending the life (and obtaining accurate readings) of a battery in other devices, such as, for example and without limitation, a smartphone regardless of whether the smartphone is part of an analyte monitoring system.

What is claimed is:

1. An analyte monitoring system comprising:
a display device; and
a transceiver including a battery and configured to (i) perform a battery level reading function to determine a battery level of the battery and (ii) convey to the display device one or more of the determined battery level and a battery level alert, alarm, or notification generated based the determined battery level, wherein the transceiver is configured to time performance of the battery level reading function such that it does not occur at the same time as a period of high current demand on the battery.

2. The analyte monitoring system of claim 1, wherein timing performance of the battery level reading function such that it does not occur at the same time as the period of high current demand on the battery includes not performing the battery level reading function at the same time as the transceiver is executing concurrently a threshold number of functions or more.

3. The analyte monitoring system of claim 1, wherein timing performance of the battery level reading function such that it does not occur at the same time as the period of high current demand on the battery includes not performing the battery level reading function at the same time as the transceiver is executing one or more high current demand functions.

4. The analyte monitoring system of claim 3, further comprising an analyte sensor, wherein the one or more high current demand functions include communicating with the analyte sensor.

5. The analyte monitoring system of claim 1, wherein timing performance of the battery level reading function such that it does not occur at the same time as the period of high current demand on the battery includes not performing the battery level reading function at the same time as the transceiver is executing concurrently functions that have a combined current demand higher than a current demand threshold.

6. The analyte monitoring system of claim 1, wherein timing performance of the battery level reading function such that it does not occur at the same time as the period of high current demand on the battery comprises using one or more software semaphores.

7. The analyte monitoring system of claim 1, wherein performing the battery level reading function includes sampling the voltage of the battery.

8. The analyte monitoring system of claim 1, wherein the transceiver is further configured to time performance of the battery level reading function such that it does not occur soon after the period of high current demand on the battery.

9. The analyte monitoring system of claim 1, wherein the transceiver is further configured to perform the battery level reading function in a time frame isolated from the period of high current demand such that enough time passes after the period of high current demand for the battery level reading to be accurate and not reflect a temporary voltage drop due to the high current demand.

10. An analyte monitoring system comprising:
an analyte sensor;
a display device; and
a transceiver including a battery and configured to (1) execute functions including communicating with the analyte sensor and communicating with the display device, (2) be capable of executing concurrently two or more of the functions, and (3) avoid executing concurrently one or more of:
(a) a number of functions greater than a maximum number of functions;
(b) functions including one or more high current demand functions; and
(c) two or more functions that would have a combined current demand higher than a current demand threshold.

11. The analyte monitoring system of claim 10, wherein the transceiver is configured to avoid executing concurrently the function of communicating with the analyte sensor and another of the functions.

12. The analyte monitoring system of claim 10, wherein the transceiver is configured to avoid executing concurrently the functions of communicating with the analyte sensor and communicating with the display device.

13. The analyte monitoring system of claim 10, wherein the transceiver is configured to (i) perform a battery level reading function to determine a battery level of the battery and (ii) convey to the display device one or more of the determined battery level and a battery level alert, alarm, or notification generated based the determined battery level, wherein the transceiver is configured to time performance of the battery level reading function such that it does not occur at the same time as a period of high current demand on the battery.

14. A method comprising:
using a transceiver to perform a battery level reading function to determine a battery level of a battery of the transceiver;
using the transceiver to convey to a display device one or more of the determined battery level and a battery level alert, alarm, or notification generated based the determined battery level; and
using the transceiver to time performance of the battery level reading function such that it does not occur at the same time as a period of high current demand on the battery.

15. A method comprising:
using transceiver to execute functions;
using the transceiver to concurrently execute two or more of the functions; and
using the transceiver to avoid executing concurrently one or more of:
(a) a number of functions greater than a maximum number of functions;
(b) functions including one or more high current demand functions; and
(c) two or more functions that would have a combined current demand higher than a current demand threshold.

16. The method of claim 15, wherein the functions include communicating with an analyte sensor and communicating with a display device.
